(19) United States
(12) Patent Application Publication
(10) Pub. No.: US 2011/0224560 A1
(43) Pub. Date: Sep. 15, 2011

(54) SPHYGMOMANOMETER CUFF AND SPHYGMOMANOMETER PROVIDED WITH THE SAME

(75) Inventors: Masashi KITAMURA, Minato-ku (JP); Minoru TANIGUCHI, Kyoto-shi (JP); Yoichiro WATANABE, Kyoto-shi (JP); Lan-Lan WANG, Kyoto-shi (JP); Yuuichi NORO, Matsusaka-shi (JP)

(73) Assignee: OMRON HEALTHCARE CO., LTD., Kyoto-shi (JP)

(21) Appl. No.: 12/884,229

(22) Filed: Sep. 17, 2010

Related U.S. Application Data
(63) Continuation of application No. PCT/JP2009/054859, filed on Mar. 13, 2009.

(30) Foreign Application Priority Data
Mar. 19, 2008 (JP) 2008-071932

(51) Int. Cl. A61B 5/0225

(52) U.S. Cl. 600/493

(57) ABSTRACT

A sphygmomanometer cuff is provided with an upper arm support stand arranged to support an upper arm, and an arm band arranged to be wound around the upper arm. The upper arm support stand includes an upper arm support surface arranged to support the upper arm while the upper arm is placed thereon, a winding mechanism arranged to pull one end of the arm band in the winding direction so as to wind a portion of the arm band close to the one end, and a hook arranged to engage a portion of the arm band close to another end pulled out from the winding mechanism against a pulling force applied by the winding mechanism. The upper arm supported on the upper arm support stand is tightened by the upper arm support surface and the portion of the arm band pulled out from the winding mechanism in a state where the portion of the arm band close to the other end is engaged with the hook. With such a configuration, the sphygmomanometer cuff is capable of being easily attached to the upper arm serving as a measurement site having a difference in size as well as repeating reliable winding around the upper arm in every measurement.

### BACKGROUND OF THE INVENTION

1. Field of the Invention

The present invention relates to a sphygmomanometer cuff that can be attached to an upper arm to be used for measuring a blood pressure value (hereinafter, also simply referred to as a cuff) and a sphygmomanometer including such a cuff.

2. Description of the Related Art

In general, when measuring a blood pressure value, a cuff accommodating a fluid bag for compressing an artery positioned inside a living body is wound around a body surface of the living body, the wound fluid bag is expanded and contracted so as to detect an arterial blood pressure pulse wave generated in the artery, and the blood pressure value is measured based on the detected pulse wave. The cuff is a band-shaped structure having an inner cavity capable of being wound around part of the living body, which is utilized for measuring the blood pressure value by injecting a fluid such as gas and liquid into the inner cavity so as to expand and contract the fluid bag. It should be noted that an upper arm is generally and frequently adopted as a measurement site of the living body to which the cuff is attached, and the cuff particularly wound around an arm to be used is also called an arm band or a manchette.

A sphygmomanometer is roughly divided based on a configuration difference into a sphygmomanometer in which the cuff and a main body are integrated, and a sphygmomanometer in which the cuff and the main body are separated from each other. In the sphygmomanometer in which the cuff and the main body are integrated, a hollow opening portion into which a measurement site is to be inserted is formed in the main body, and the cuff is provided with the main body so as to surround this hollow opening portion. Meanwhile, in the sphygmomanometer in which the cuff and the main body are separated from each other, an expanding/contracting mechanism for expanding and contracting an air bladder serving as the fluid bag accommodated in the cuff (normally a pressurization pump, an exhaust valve, or the like) is provided in the main body, and the air bladder accommodated in the cuff and the expanding/contracting mechanism provided in the main body are connected to each other by a flexible air tube.

For example, Japanese Unexamined Patent Publication No. 2005-237427 discloses a sphygmomanometer in which the cuff and the main body are integrated. In the sphygmomanometer in which the cuff and the main body are integrated, a drive mechanism and the like for winding the cuff can be relatively easily provided in the main body with which the cuff is provided. Therefore, only by inserting the measurement site into the hollow opening portion provided in the main body, attachment of the cuff and measurement of the blood pressure value can be automatically performed. Thus, it can be said that the sphygmomanometer in which the cuff and the main body are integrated is excellent in that the cuff can be highly easily attached and detached. On the other hand, in the sphygmomanometer in which the cuff and the main body are integrated, there are problems including a problem that the device is necessarily increased in size, a problem that the device configuration is complicated so as to increase manufacturing cost, and a problem that since measurable size of the measurement site is limited by the device configuration, a plurality of devices having different sizes of the hollow opening portions are required to be prepared in order to be widely fit for a variety of people in consideration of the size of the measurement site.

Meanwhile, in the sphygmomanometer in which the cuff and the main body are separated from each other, by separating the cuff from the main body, the cuff can be relatively small with an excellent accommodating property, and the sphygmomanometer has favorable usability under various use environments. However, in the sphygmomanometer in which the cuff and the main body are separated from each other, an attachment task of the cuff must be performed by the hands of a human such as a subject. Thus, reliable winding of the cuff around the measurement site is not always repeated in every measurement. In order to more precisely and stably measure the blood pressure value, the cuff is required to be reliably wound around the measurement site. In this regard, the sphygmomanometer in which the cuff and the main body are separated still has room for improvement.

Therefore, variously formed cuffs have been conventionally proposed for reliably winding the cuff around the measurement site with favorable repetition in the sphygmomanometer in which the cuff and the main body are separated from each other. For example, Japanese Unexamined Patent Publication No. S61-238229 and Japanese Unexamined Patent Publication No. 2002-209858 and the like disclose a cuff accommodating a flexible member called a curler inside in addition to the air bladder. The curler is accommodated inside the cuff for maintaining an annular form of the cuff. The curler is annularly wrapped around the outer side of the air bladder and arranged inside the cuff, so that the cuff is formed to be elastically deformable in the radial direction. In the cuff provided with such a curler, the air bladder is fixed while being pushed toward the measurement site by the curler with proper pressing force after attachment. Thus, reliable fixation of the air bladder to the measurement site is repeated.

However, even in the cuff accommodating the curler, there is a problem that a winding task thereof is troublesome in comparison to the above sphygmomanometer in which the cuff and the main body are integrated. This is due to the fact that the curler is shaped so that the curler during a time of non-attachment has a more reduced diameter than the measurement site in order to reliably press the air bladder onto the measurement site at a time of attaching the cuff. That is, at a time of attaching the cuff, the curler in a reduced diameter state is required to be once pushed and extended so as to be attached to the measurement site. This pushing and extending task causes problems. Particularly, in a sphygmomanometer for domestic use, the subject himself/herself is required to wind the cuff around one of his/her arms. Thus, the subject is only able to use the other hand at a time of attachment. Therefore, the subject is required to get used to, to some extent, a task of pushing and extending the cuff in the reduced diameter state and attaching the cuff to the arm with a single hand. As well as the sphygmomanometer in which the cuff and the main body are integrated, since the measurable size of the measurement site is limited by the size of the cuff, a plurality of cuffs having different sizes are required to be prepared in order to be widely fit for a variety of people in consideration of the size of the measurement site.

As described above, in the conventional sphygmomanometer cuff, it cannot be said that easy attachment and the repetition of winding are always realized at the same time, and the sphygmomanometer cuff still has room for improvement in this regard. The conventional sphygmomanometer cuff has the problem that the measurable size of the upper arm serving as the measurement site is limited, and improvement is also required in this regard.

### SUMMARY OF THE INVENTION

Therefore, in view of solving the above problems, preferred embodiments of the present invention provide a sphygmomanometer cuff capable of being easily attached to the upper arm serving as the measurement site having a wide difference in size and repeating reliable winding around the upper arm in every measurement, and a sphygmomanometer provided with such a cuff.

A sphygmomanometer cuff according to a preferred embodiment of the present invention is attached to an upper arm to be used for measuring a blood pressure value, and includes an upper arm support stand arranged to support the upper arm while being mounted on a mount surface, and a long-band shaped arm band capable of being wound around the upper arm in a state where the upper arm is supported on the upper arm support stand. The arm band includes a fluid bag arranged to compress the upper arm while being expanded, and the upper arm support stand includes an upper arm support surface arranged to support the upper arm while the upper arm is placed thereon, a winding mechanism capable of pulling one end in the longitudinal direction of the arm band in the winding direction so as to wind a portion of the arm band close to the one end, and an engaging portion capable of engaging a portion of the arm band close to the other end in the longitudinal direction pulled out from the winding mechanism against pulling force by the winding mechanism. In the sphygmomanometer cuff, in a state where the portion of the arm band close to the other end is engaged with the engaging portion, the upper arm supported on the upper arm support stand is capable of being tightened by the upper arm support surface and the portion of the arm band pulled out from the winding mechanism.

In the sphygmomanometer cuff according to a preferred embodiment of the present invention, preferably, the upper arm support surface is provided in an upper portion of the upper arm support stand while being inclined so that the upper arm support surface is inclined in a state where the upper arm support stand is mounted on the mount surface.

In the sphygmomanometer cuff according to a preferred embodiment of the present invention, preferably, the upper arm support stand further includes an elbow rest on which an elbow is mountable in a state where the upper arm is placed on the upper arm support surface.

In the sphygmomanometer cuff according to a preferred embodiment of the present invention, preferably, the winding mechanism includes a winding roller to which the one end of the arm band is fixed, and a bias portion arranged to bias the winding roller in the winding direction.

In the sphygmomanometer cuff according to a preferred embodiment of the present invention, preferably, the winding mechanism is arranged inside the upper arm support stand, and in that case, preferably, the upper arm support stand further includes an arm band pull-out port from which the portion of the arm band close to the other end is pulled out.

In the sphygmomanometer cuff according to a preferred embodiment of the present invention, preferably, the engaging portion and the arm band pull-out port are arranged so as to sandwich the upper arm support surface.

In the sphygmomanometer cuff according to a preferred embodiment of the present invention, preferably, in a state where the arm band is wound by the winding mechanism as much as possible, the portion of the arm band close to the other end is pulled out from the arm band pull-out port by a predetermined length.

In the sphygmomanometer cuff according to a preferred embodiment of the present invention, preferably, the upper arm support stand further includes a guide portion arranged to guide the portion of the arm band close to the other end pulled out from the arm band pull-out port in the state where the arm band is wound by the winding mechanism as much as possible.

In the sphygmomanometer cuff according to a preferred embodiment of the present invention, preferably, the upper arm support stand further includes a locking mechanism arranged to prevent pull-out of the arm band from the winding mechanism.

In the sphygmomanometer cuff according to a preferred embodiment of the present invention, preferably, the locking mechanism is actuated in connection with engagement of the arm band with the engaging portion and in that case, preferably, the pull-out of the arm band from the winding mechanism is prevented only in a state where the arm band is engaged with the engaging portion.

In the sphygmomanometer cuff according to a preferred embodiment of the present invention, preferably, a hole portion is provided in the portion of the arm band close to the other end, and in that case, preferably, the engaging portion is formed by a hook portion capable of being hung on the hole portion.

In the sphygmomanometer cuff according to a preferred embodiment of the present invention, preferably, the hole portion has a substantially circular shape in a plan view, and in that case, preferably, the hook portion has a substantially cylindrical shape.

A sphygmomanometer according to yet another preferred embodiment of the present invention is provided with any of the above sphygmomanometer cuffs, an expanding/contracting mechanism arranged to expand and contract the fluid bag, a pressure detector arranged to detect a pressure in the fluid bag, and a blood pressure value calculating unit arranged to calculate a blood pressure value based on pressure information detected by the pressure detector.

According to a preferred embodiment of the present invention, the sphygmomanometer cuff capable of being easily attached to the upper arm serving as the measurement site having a wide difference in size and repeating reliable winding around the upper arm in every measurement and the sphygmomanometer can be obtained. Therefore, by measuring the blood pressure value by using the sphygmomanometer cuff and the sphygmomanometer including such a cuff, the blood pressure value can be precisely and stably measured and the measurement can be performed irrespective of size of the upper arm.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a view showing an outer appearance structure of a sphygmomanometer according to a preferred embodiment of the present invention.

FIG. 2 is a functional block configuration diagram of the sphygmomanometer in FIG. 1.

FIG. 3 is a flowchart showing a flow of blood pressure value measurement processing of the sphygmomanometer in FIG. 1.

FIG. 4 is a right side view of a cuff shown in FIG. 1.

FIG. 5 is a left side view of the cuff shown in FIG. 1.

FIG. 6 is a perspective view showing an outer appearance of an accommodating state of the cuff in FIG. 1.

FIG. 7 is a vertically sectional view of the cuff shown in FIG. 1.

FIG. 8 is a sectional view of the cuff along the line VIII-VIII in FIG. 7.

FIG. 9 is a sectional view of the cuff along the line IX-IX in FIG. 7.

FIG. 10 is a sectional view of the cuff along the line X-X in FIG. 7.

FIG. 11 is a sectional view of the cuff along the line XI-XI in FIG. 7.

FIG. 12 is an exploded perspective view showing an assembling structure of a case body of the cuff in FIG. 1.

FIG. 13 is a perspective view showing a state where the sphygmomanometer cuff in FIG. 1 is attached to an upper arm.

FIG. 14 is a sectional view showing a state where the sphygmomanometer cuff in FIG. 1 is attached to the upper arm.

### DETAILED DESCRIPTION OF THE PREFERRED EMBODIMENTS

Preferred embodiments of the present invention will be hereinafter described in detail with reference to the drawings. It should be noted that a sphygmomanometer in the preferred embodiments below preferably is a so-called upper arm type sphygmomanometer in which an upper arm is adopted as a measurement site, and a cuff provided in the sphygmomanometer is intended to be attached to the upper arm.

### First Preferred Embodiment

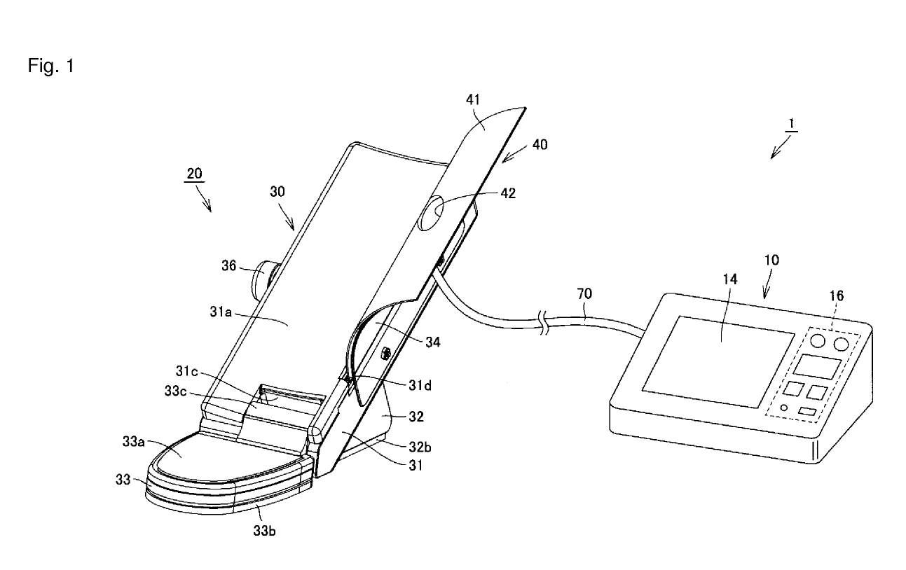

FIG. 1 is a view showing an outer appearance structure of the sphygmomanometer according to a preferred embodiment of the present invention. Firstly, with reference to FIG. 1, the outer appearance structure of the sphygmomanometer in the present preferred embodiment will be described.

As shown in FIG. 1, the sphygmomanometer 1 in the present preferred embodiment is provided mainly with a main body 10, a cuff 20, and an air tube 70. The main body 10 is mounted on a surface of a table or the like to be used during measurement, and a display unit 14 and an operation unit 16 are provided on an upper surface thereof. The cuff 20 is attached to the upper arm to be used during measurement while being mounted on the surface of a table or the like, and including an upper arm support stand 30 and an arm band 40. The air tube 70 is a member arranged to couple the main body 10 and the cuff 20 which are separated and formed by a flexible tube.

The upper arm support stand 30 of the cuff 20 mainly includes a case body 31 including an upper arm support surface 31a onto which an upper arm is placed during measurement, a stand portion 32 provided on the lower side of the case body 31, an elbow rest 33 provided on the front side of a lower end of the case body 31, and a guide portion 34 additionally provided on one side surface of the case body 31. The arm band 40 of the cuff 20 preferably includes a long-band shaped member, a portion thereof close to one end in the longitudinal direction is positioned inside the case body 31, and a portion thereof close to the other end is pulled out from an arm band pull-out port 31d provided in an end of the case body 31 on the upper surface side to the outside of the case body 31. It should be noted that a detailed structure of the cuff 20 will be described later.

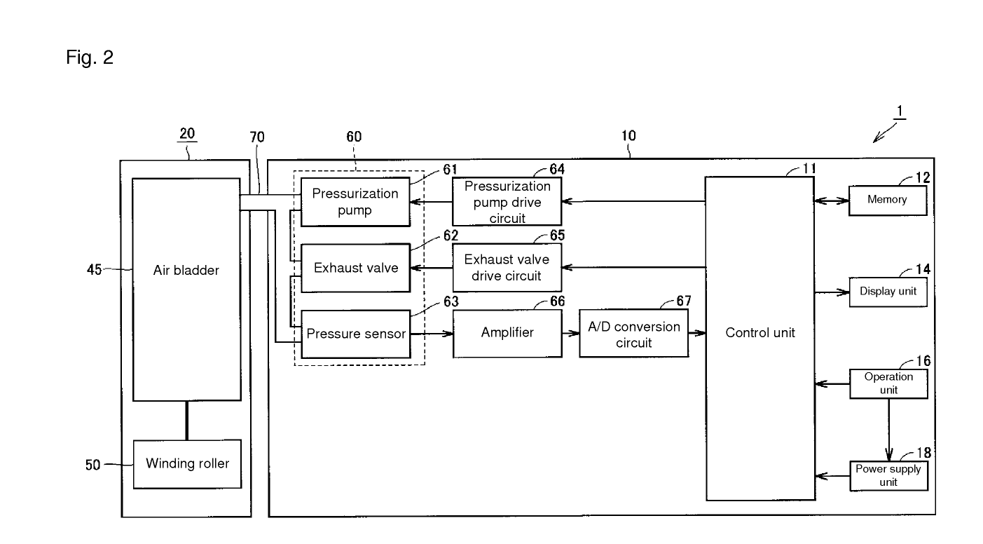

FIG. 2 is a functional block configuration diagram of the sphygmomanometer in FIG. 1. Next, with reference to FIG. 2, a functional block configuration of the sphygmomanometer in the present preferred embodiment will be described.

As shown in FIG. 2, the main body 10 includes a control unit 11, a memory unit 12, a power supply unit 18, a pressurization pump 61, an exhaust valve 62, a pressure sensor 63, a pressurization pump drive circuit 64, an exhaust valve drive circuit 65, an amplifier 66, and an A/D (Analog/Digital) conversion circuit 67 in addition to the display unit 14 and the operation unit 16. Meanwhile, the cuff 20 includes an air bladder 45 and a winding roller 50.

The control unit 11 preferably includes, for example, a CPU (Central Processing Unit) that is arranged and programmed to control the entire sphygmomanometer 1. The memory unit 12 preferably includes, for example, a ROM (Read-Only Memory) and a RAM (Random-Access Memory) arranged to store a program to be executed by the control unit 11 and the like to perform procedures to measure a blood pressure value and store a measurement result and the like. The display unit 14 preferably includes, for example, a LCD (Liquid Crystal Display) to display the measurement result and the like. The operation unit 16 receives an operation of a subject or the like and inputs a command from the outside to the control unit 11 and the power supply unit 18. The power supply unit 18 supplies electric power as a power supply to the control unit 11.

The control unit 11 inputs control signals to drive the pressurization pump 61 and the exhaust valve 62 to the pressurization pump drive circuit 64 and the exhaust valve drive circuit 65, and inputs the blood pressure value as the measurement result to the memory unit 12 and the display unit 14. The control unit 11 calculates the blood pressure value of the subject based on a pressure value detected by the pressure sensor 63. Therefore, the control unit 11 also functions as a blood pressure value calculating unit. The blood pressure value acquired by this control unit 11 is inputted to the memory unit 12 and the display unit 14 as the measurement result. It should be noted that the sphygmomanometer 1 may have an output unit arranged to output the blood pressure value as the measurement result to an external device (such as a PC (Personal Computer) and a printer). For example, a serial communication line, a writing device to various recording media and the like can be utilized as the output unit.

The air bladder 45 preferably includes a fluid bag arranged to compress the upper arm in an attachment state, which is connected to an air system component 60 described later via the air tube 70. The air bladder 45 is accommodated in the arm band 40. The winding roller 50 defines a portion of a winding mechanism arranged to wind the arm band 40, and is provided in the upper arm support stand 30.

The pressurization pump drive circuit 64 controls an operation of the pressurization pump 61 based on the control signal inputted from the control unit 11. The exhaust valve drive circuit 65 controls an open/close operation of the exhaust valve 62 based on the control signal inputted from the control unit 11. The amplifier 66 amplifies and inputs an output value of the pressure sensor 63 serving as a pressure detector to the A/D conversion circuit 67. The A/D conversion circuit 67 converts an analog signal inputted from the amplifier 66 into a digital signal and inputs the signal to the control unit 11.

The pressurization pump 61 supplies the air into an inner cavity of the air bladder 45, and an operation thereof is controlled by the pressurization pump drive circuit 64. The exhaust valve 62 maintains pressure inside the air bladder 45 (hereinafter, also referred to as the cuff pressure) and opens space inside the air bladder 45 to the outside, and an operation thereof is controlled by the exhaust valve drive circuit 65. The pressure sensor 63 inputs an output value corresponding to the pressure inside the air bladder 45 to the amplifier 66. It should be noted that among these constituent elements, the pressurization pump 61, the exhaust valve 62 and the pressure sensor 63 correspond to the air system component 60, and particularly the pressurization pump 61 and the exhaust valve 62 correspond to an expanding/contracting mechanism arranged to expand and contract the air bladder 45.

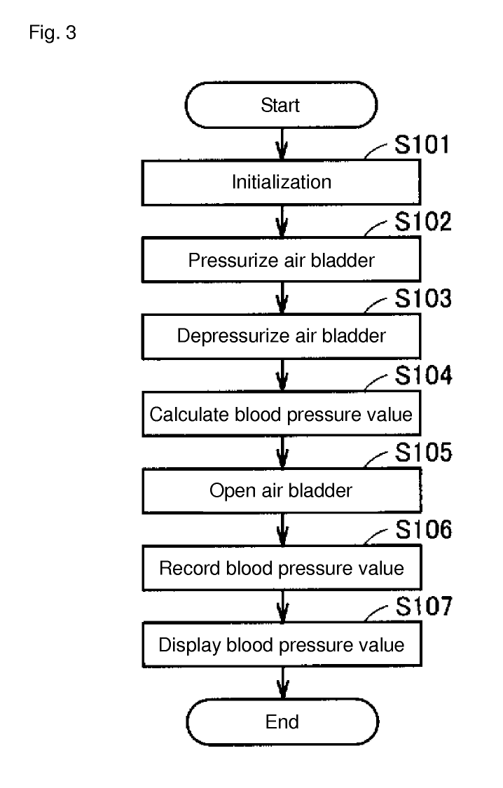

FIG. 3 is a flowchart showing a flow of blood pressure value measurement processing of the sphygmomanometer in FIG. 1. Next, with reference to FIG. 3, the flow of the blood pressure value measurement processing in the sphygmomanometer in the present preferred embodiment will be described. A program according to this flowchart is preliminarily stored in the memory unit 12 shown in FIG. 2, and the control unit 11 reads out and executes this program from the memory unit 12, so that the blood pressure value measurement processing is executed.

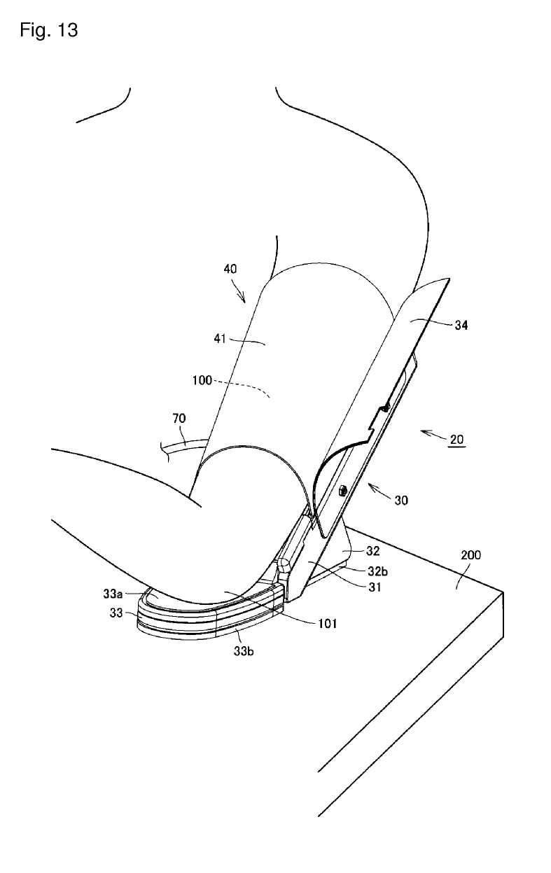

Firstly, as shown in FIG. 3, when the subject with the cuff 20 attached to an upper arm 100 (refer to FIG. 13 and the like) operates the operation unit 16 of the sphygmomanometer 1 and inputs a command to turn ON power, the electric power as the power supply is supplied from the power supply unit 18 to the control unit 11, so that the control unit 11 is driven and initialization of the sphygmomanometer 1 is performed (Step S101). Next, the control unit 11 starts pressurization of the air bladder 45 in order to measure the blood pressure value (Step S102). Specifically, the control unit 11 drives the pressurization pump 61 and increases the cuff pressure so as to pressurize the air bladder 45 to obtain a predetermined cuff pressure.

Next, the control unit 11 starts slow depressurization of the air bladder 45 to measure the blood pressure value (Step S103). Specifically, the control unit 11 stops driving the pressurization pump 61, and then gradually opens the exhaust valve 62 while controlling an open amount of the exhaust valve 62. At that time, the control unit 11 acquires a change in the cuff pressure detected by the pressure sensor 63.

Next, the control unit 11 calculates the blood pressure value based on the change in the cuff pressure obtained in the slow depressurization process (Step S104). Successively, the control unit 11 opens the air bladder 45 (Step S105). Specifically, the control unit 11 completely opens the exhaust valve 62 so as to exhaust the air in the air bladder 45 to the outside.

Next, the control unit 11 outputs the blood pressure value obtained in Step S104 to the memory unit 12 and the display unit 14 and the blood pressure value is stored in the memory unit 12 as the measurement result (Step S106). The blood pressure value as the measurement result is displayed in the display unit 14 (Step S107). The display unit 14 displays a systolic blood pressure value and a diastolic blood pressure value, for example, as numerical values. The sphygmomanometer 1 is in a standby state after recording and displaying these blood pressure values, and stops the supply of the electric power as the power supply upon input of a command to turn OFF power by the subject with the operation unit 16.

It should be noted that the measurement method described above is based on a so-called depressurization measurement method of detecting a pulse wave during depressurization of the air bladder and calculating the blood pressure value, but as a matter of course, a so-called pressurization measurement method of detecting the pulse wave during pressurization of the air bladder and calculating the blood pressure value may be adopted.

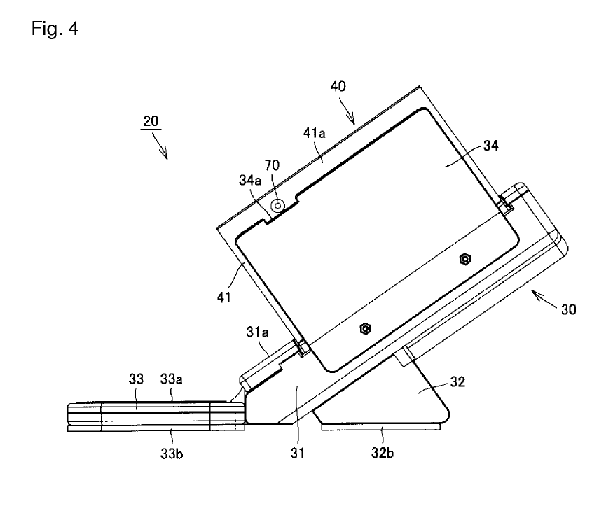

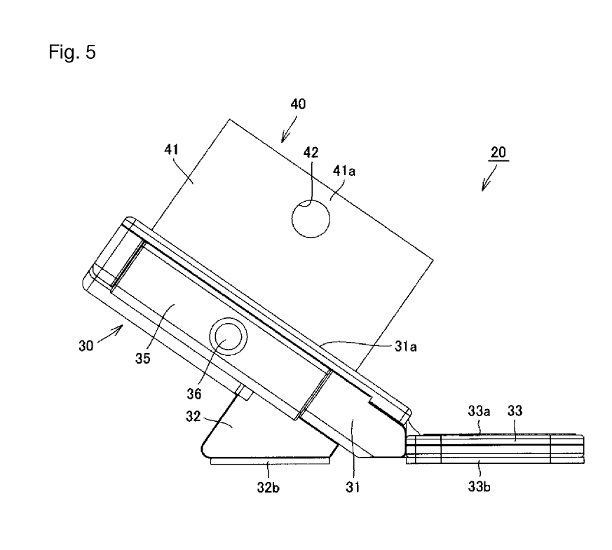

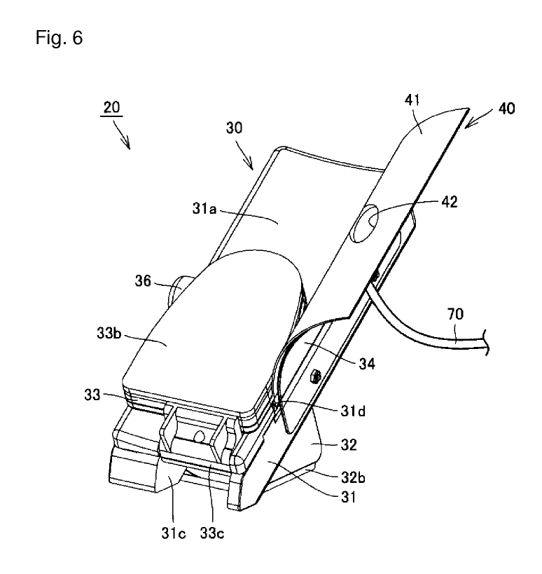

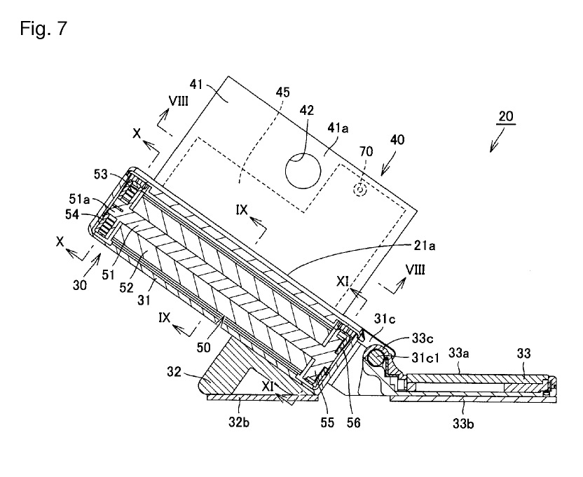

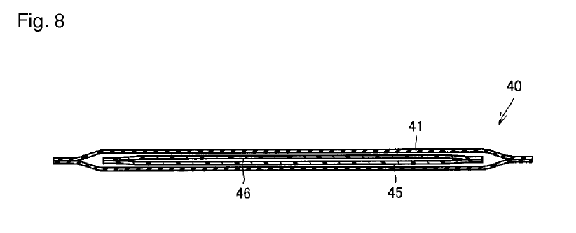

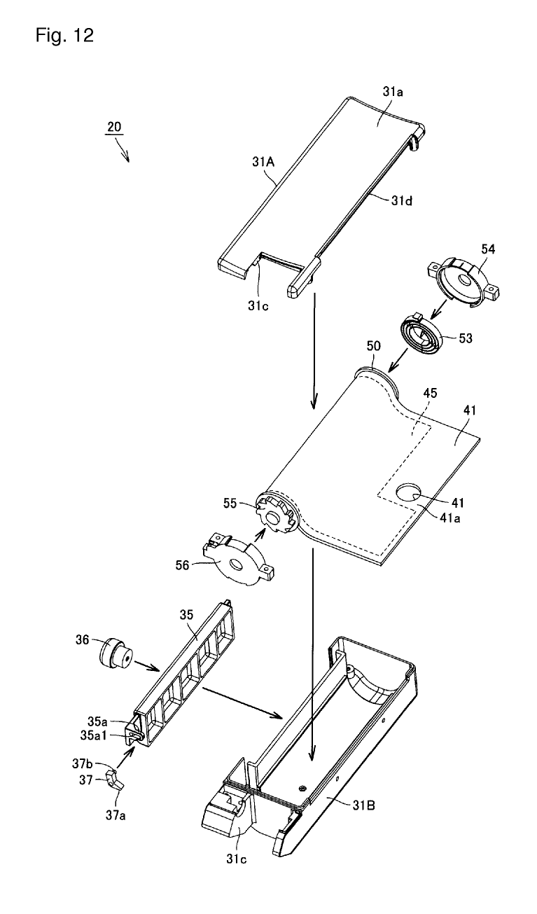

FIGS. 4 and 5 are right and left side views of the cuff in FIG. 1, and FIG. 6 is a perspective view showing an outer appearance of an accommodating state of the cuff in FIG. 1. FIG. 7 is a vertically sectional view of the cuff in FIG. 1, and FIGS. 8 to 11 are sectional views of the cuff respectively along the lines VIII-VIII, IX-IX, X-X and XI-XI in FIG. 7. FIG. 12 is an exploded perspective view showing an assembly structure of the case body of the cuff in FIG. 1. Next, with reference to FIGS. 4 to 12 as well as FIG. 1, a detailed structure of the cuff in the present preferred embodiment will be described.

With reference to FIGS. 1, 4 and 5, as described above, the cuff 20 in the present preferred embodiment is preferably provided with the upper arm support stand 30 and the arm band 40. The case body 31 of the upper arm support stand 30 preferably has a substantially rectangular parallelepiped shape, and has the upper arm support surface 31a on an upper surface thereof. In more detail, as shown in FIG. 12, the case body 31 is formed by a case 31A with an upper surface opening, and a lid 31B for opening and closing the upper surface opening of the case 31A, and formed into a box shape by assembling the case 31A and the lid 31B. The winding roller 50 described later and the like are accommodated inside this box-shaped case body 31. The upper arm support surface 31a is provided on an upper surface of the lid 31B, and has a concave shape slightly curved in the width direction (that is, the parallel direction to the short side of the case body 31) so as to be fit for the upper arm.

The stand portion 32 is provided on the lower side of the upper arm support stand 30, and the case body 31 is supported by the stand portion 32. The upper arm support surface 31a is provided while being inclined relative to a lower surface of the stand portion 32 so that the upper arm support surface is inclined by a predetermined angle in a state where the upper arm support stand 30 is mounted on the horizontal mount surface. A leg portion 32b for preventing that the upper arm support stand 30 mounted on the mount surface slides on the mount surface is attached to the lower surface of the stand portion 32. This leg portion 32b preferably is, for example, defined by a high frictional member such as rubber.

The elbow rest stand 33 is positioned on the front side of the lower end of the case body 31 and on the front side of the stand portion 32. The elbow rest stand 33 includes an elbow mount surface 33a on an upper surface thereof, and the elbow mount surface 33a is arranged so as to be substantially continuous to the upper arm support surface 31a provided in the case body 31. A leg portion 33b arranged to prevent the upper arm support stand 30 mounted on the mount surface from sliding on the mount surface is attached to a lower surface of the elbow rest stand 33 as well as the lower surface of the stand portion 32. This leg portion 33b preferably is also defined by the high frictional member such as rubber, for example.

As shown in FIGS. 1 and 6, the elbow rest stand 33 is rotatably coupled to the case body 31. Therefore, the cuff 20 is in a use state shown in FIG. 1, or in the accommodating state shown in FIG. 6. Specifically, a cutout portion 31c is provided in the lower end of the case body 31, a shaft portion 31c1 is arranged so as to hang over both side surfaces of the cutout portion 31c from these both side surfaces (refer to FIG. 7), and a coupling portion 33c fitted over this shaft portion 31c1 to rotatably couple the elbow rest stand 33 to the case body 31 is provided in a rear end of the elbow rest stand 33. In the accommodating state shown in FIG. 6, the elbow rest stand 33 is rotated and moved so that the upper arm support surface 31a of the case body 31 faces the elbow mount surface 33a of the elbow rest stand 33, and the elbow rest stand 33 is arranged on the case body 31. Thereby, an outer shape of the cuff 20 is made compact to be suitable for accommodating.

As shown in FIGS. 1 and 4, the arm band pull-out port 31d is provided an end of the case body 31 on the upper surface side and on the right side surface side. This arm band pull-out port 31d is preferably defined by a long and thin slit extending in the direction in which the upper arm support surface 31a extends (that is, the direction parallel or substantially parallel to the long side of the case body 31). The portion of the arm band 40 close to the other end 42a in the longitudinal direction is pulled out from the arm band pull-out port 31d.

The guide portion 34 is provided in parallel to the arm band pull-out port 31d on a right side surface of the case body 31 in which the arm band pull-out port 31d is provided so as to protrude upward over the upper arm support surface 31a. The guide portion 34 is preferably defined by a curved plate member, and a cutout is provided at a predetermined position on an upper end thereof, so that a hanging portion 34a is provided. The guide portion 34 is a support frame for generating a state where the portion of the arm band 40 which is pulled out from the arm band pull-out port 31d stands up (that is, for generating a state where the portion of the arm band 40 on the other end 41a side extends upward). In more detail, the portion of the arm band 40 which is pulled out from the arm band pull-out port 31d stands up while hanging a portion of the air tube 70 which is connected to the arm band 40 onto the hanging portion 34a of the guide portion 34.

As shown in FIGS. 1 and 5, a sliding member 35 movable in the up and down direction relative to the case body 31 is attached to a left side surface of the case body 31. The sliding member 35 defines a portion of a locking mechanism arranged to prevent pull-out of the arm band 40 from the winding roller 50, and is arranged to be movable in the up and down direction by a predetermined distance while being guided by a guide groove (not shown) provided in the case body 31. A hook 36 protruding from a main surface of the sliding member 35 toward the side is provided on the main surface. The hook 36 corresponds to an engaging portion arranged to engage a hole 42 provided in the arm band 40 described later, and a shape thereof is substantially cylindrical.

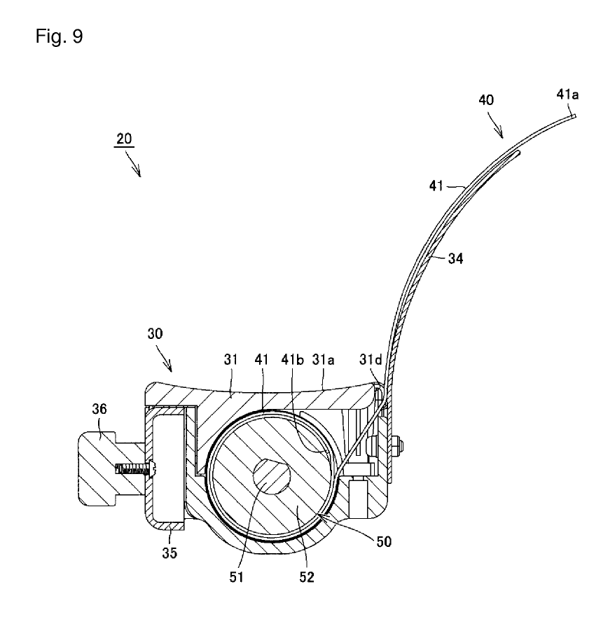

As shown in FIG. 7, the winding roller 50 is accommodated inside the case body 31. This winding roller 50 is rotatably axially supported by the case body 31. One end 41b in the longitudinal direction of the arm band 40 (refer to FIG. 9) is fixed to the winding roller 50, so that the portion of the arm band 40 close to the one end 41b is capable of being wound around the winding roller 50.

As shown in FIGS. 7 and 8, the arm band 40 has a bag-shaped cover 41 having space inside, and the air bladder 45 accommodated in the bag-shaped cover 41. The bag-shaped cover 41 and the air bladder 45 both have substantially rectangular shapes in a plan view while being developed, and portions of the bag-shaped cover and the air bladder are capable of being wound by the winding roller 50.

The bag-shaped cover 41 is preferably formed by a cloth made of synthetic resin of polyamide (PA), polyester or the like, for example, and includes an inner cover portion forming an inner peripheral side portion in contact with the upper arm when the arm band 40 is wound around the upper arm, and an outer cover portion forming an outer peripheral side portion not in contact with the upper arm when the arm band 40 is wound around the upper arm. The inner cover portion of the bag-shaped cover 41 is preferably formed by a member excellent in stretchability, and the outer cover portion of the bag-shaped cover 41 is preferably formed by a member having stretchability less than the inner cover portion, for example. The inner cover portion and the outer cover portion of the bag-shaped cover 41 are combined to each other by welding, sewing or the like, for example. The bag-shaped cover 41 has the hole 42 in the substantial center in the width direction in the portion thereof in the longitudinal direction close to the other end 41a. This hole 42 is arranged to hang the hook 36 of the sliding member 35 attached to the left side surface of the case body 31, and preferably has a substantially circular shape in a plan view so as to pass through the inner cover portion and the outer cover portion.

The air bladder 45 is preferably formed by a bag-shaped member made of a resin sheet. The air bladder 45 is preferably formed into a bag shape by overlapping an inner sheet to be positioned on the upper arm side when the arm band 40 is wound around the upper arm, and an outer sheet to be positioned on the outer side of the inner sheet when the arm band 40 is wound around the upper arm, and welding peripheral edges thereof. The air bladder has expanding/contracting space 46 inside. This expanding/contracting space 46 is connected to the air tube 70 via a nipple (not shown), and pressurization and depressurization thereof are performed by the expanding/contracting mechanism. It should be noted that the material for the resin sheet forming the air bladder 45 may be of any kind as long as it excels in stretchability and the air does not leak out from the expansion/contraction space after welding. In such a view, favorable materials for the resin sheet include ethylene-polyvinyl acetate copolymer (EVA), flexible polyvinyl chloride (PVC), polyurethane (PU), polyamide (PA), and raw rubber, for example.

As shown in FIGS. 7 and 12, the winding roller 50 preferably includes a shaft 51 and a fixing member 52. The shaft 51 extends in the direction in which the upper arm support surface 31a of the case body 31 extends (that is, the direction parallel or substantially parallel to the long side of the case body 31), and has wheels on both ends thereof.

As shown in FIGS. 7 and 9, the fixing member 52 is preferably defined by a substantially cylindrical member, and fitted over the shaft 51 to be fixed. The one end 41b in the longitudinal direction of the arm band 40 is fixed at a predetermined position of a peripheral surface of the fixing member 52. The fixing member 52 is rotated by rotation of the shaft 51, so that the arm band 40 is wound and fed out by the winding roller 50.

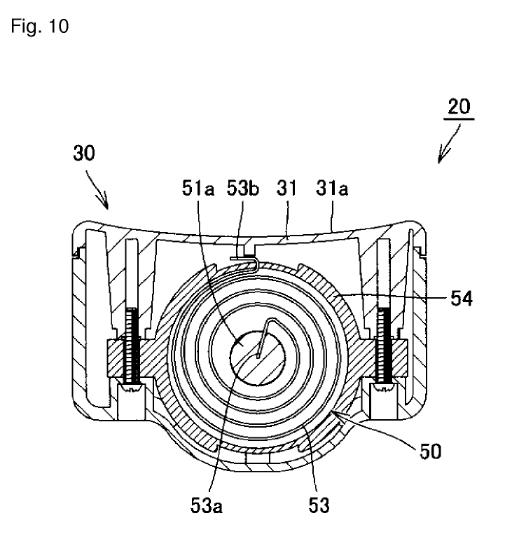

As shown in FIGS. 7 and 10, a spring 53 is attached to the wheel positioned on the upper end side of the case body 31 of the winding roller 50. This spring 53 defines a bias portion arranged to bias the winding roller 50 in the winding direction, serving as part of the winding mechanism. An inner end 53a of the spring 53 is fitted to a groove of a protruding portion 51a provided in the wheel, an outer end 53b of the spring 53 is fitted to a groove of a cap 54 fixed to the case body 31 so as to cover the spring 53. Thereby, the winding roller 50 is biased by the elastic force of the spring 53, the one end 41b of the arm band 40 is pulled in the winding direction by pulling force of the spring 53.

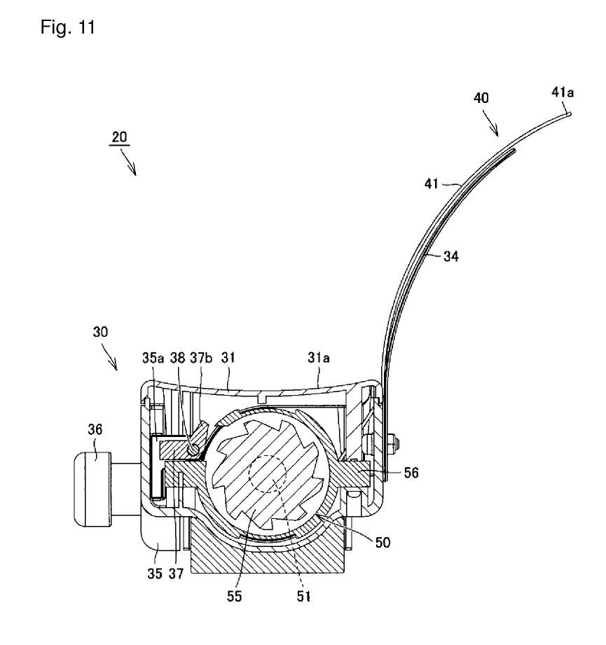

As shown in FIGS. 7 and 11, a toothed protruding portion 55 is provided in the wheel positioned on the lower end side of the case body 31 of the winding roller 50. The toothed protruding portion 55 defines a portion of the locking mechanism to prevent the pull-out of the arm band 40 from the winding roller 50, and has teeth capable of being meshed with a locking member 37 described later on a peripheral surface thereof. This toothed protruding portion 55 is covered by a cap 56 fixed to the case body 31.

As shown in FIGS. 11 and 12, the locking member 37 is arranged inside the case body 31 adjacent to the toothed protruding portion 55. The locking member 37 defines a portion of the locking mechanism to inhibit the pull-out of the arm band 40 from the winding roller 50, and includes a locking portion 37a capable of being meshed with the teeth of the toothed protruding portion 55 on a front end thereof. The locking member is rotatably axially supported by a support 38 provided in the case body 31. The locking member 37 is rotated around the support 38 in accordance with up and down movement of the sliding member 35.

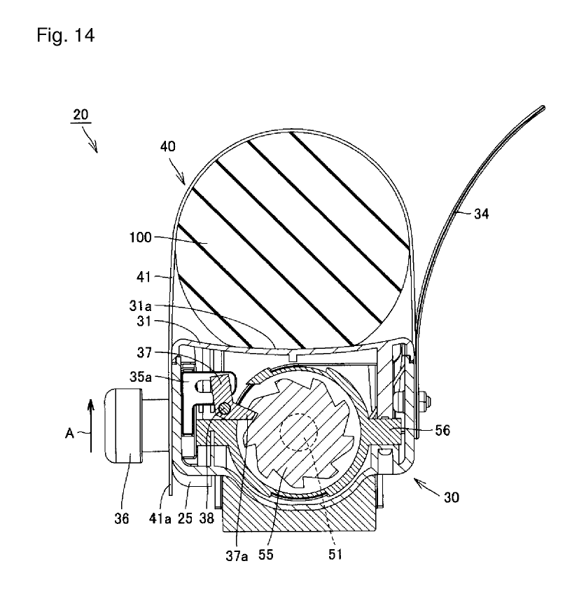

Specifically, a pin 37b to be engaged with a guide groove 35a1 of a latch portion 35a provided at a predetermined position of the sliding member 35 is provided at a predetermined position of the locking member 37. This pin 37b is guided and moved by the guide groove 35a1 in accordance with the up and down movement of the sliding member 35, so that the locking member 37 is rotated. As shown in FIG. 11, when the sliding member 35 is at a lower end in the movement direction thereof, meshing between the locking portion 37a of the locking member 37 and the teeth of the toothed protruding portion 55 is canceled. When the sliding member 35 is at an upper end in the movement direction, the locking portion 37a of the locking member 37 is meshed with the teeth of the toothed protruding portion 55 (refer to FIG. 14). Rotation of the winding roller 50 is stopped by this meshing between the locking portion 37a of the locking member 37 and the teeth of the toothed protruding portion 55, so that the arm band 40 is no more pulled out.

FIG. 13 is a perspective view showing a state where the sphygmomanometer cuff according to the present preferred embodiment is attached to the upper arm, and FIG. 14 is a sectional view showing the attachment state. It should be noted that the section shown in FIG. 14 corresponds to the above section in FIG. 11. Next, with reference to FIGS. 13 and 14, the state where the cuff according to the present preferred embodiment is attached to the upper arm will be described, and an attachment operation for realizing the attachment state will be described.

As shown in FIG. 13, at a time of attaching the cuff 20 to the upper arm 100, the cuff 20 is firstly mounted on a surface 200 of a table or the like, and the elbow rest stand 33 at an accommodating position is rotated. The upper arm 100 is placed on the upper arm support surface 31a of the upper arm support stand 30, and an elbow 101 is mounted on the elbow mount surface 33a of the elbow rest stand 33. Successively, the portion of the arm band 40 close to the other end 41a supported by the guide portion 34 to be in a standing posture is gripped with a hand which is different from a hand to be attached to the cuff 20, and wrapped around the upper arm 100 while pulling out the arm band 40. As shown in FIG. 14, the hole 42 provided in the portion of the arm band 40 close to the other end 41a is hung on the hook 36. Thus, the upper arm 100 supported on the upper arm support stand 30 is tightened by the upper arm support surface 31a and the portion of the arm band 40 pulled out from the upper arm support stand 30.

In a tightening state shown in FIGS. 13 and 14, since the one end 41b of the arm band 40 is pulled by the spring 53, the arm band 40 is exactly fit for the upper arm 100 without any gap in-between. The upper arm 100 is properly tightened by the pulling force of the spring 53. Therefore, the air bladder 45 accommodated in the arm band 40 is reliably fixed to the upper arm. As shown in FIG. 14, since the portion of the arm band 40 close to the other end 41a is hung by the hook 36, the sliding member 35 in which the hook 36 is provided is moved upward (in the arrow A direction in the figure). Thus, since the locking member 37 is rotated and the locking portion 37a is meshed with the teeth of the toothed protruding portion 55, the arm band 40 is no more pulled out. Therefore, even during a measurement operation of the blood pressure value, the air bladder 45 is expanded, so that the arm band 40 is no more pulled out from the upper arm support stand 30. Thus, a tightening length of the arm band 40 over the upper arm 100 is always maintained during the measurement operation. Therefore, a compressing force in accordance with the expansion of the air bladder 45 is efficiently applied to the upper arm 100, so that the upper arm 100 is promptly and properly compressed.

With the cuff 20 in the present preferred embodiment as described above, by highly simple operations of mounting the upper arm 100 on the upper arm support stand 30, pulling out the arm band 40 from the upper arm support stand 30 on which the upper arm 100 is mounted, and engaging the arm band 40 with the hook 36, the cuff 20 can be attached to the upper arm 100. With the cuff 20 in the present preferred embodiment, the tightening length of the arm band 40 over the upper arm 100 is always maintained during the measurement operation as described above. Therefore, by adopting the above configuration, the sphygmomanometer cuff is capable of being easily attached to the upper arm 100 and repeating reliable winding around the upper arm 100 in every measurement.

In the cuff 20 in the present preferred embodiment, the upper arm support surface 31a is inclined in a state where the upper arm support stand 30 is mounted on the surface 200 of a table or the like, and the elbow 101 is mountable on the elbow mount surface 33a of the elbow rest stand 33 in a state where the upper arm is placed on the upper arm support surface 31a of the upper arm support stand 30. Thus, a mount position of the upper arm 100 on the upper arm support surface 31a is repeated in every measurement while taking the elbow 101 mounted on the elbow rest stand 33 as a base point. With the above configuration, the elbow 101 is mounted on the elbow rest stand 33, so that the elbow rest stand 33 is sandwiched by the elbow 101 and the surface 200 of a table or the like. Thus, during the attachment operation of the arm band 40 to the upper arm 100, there is no fear that the entire cuff 20 is moved on a plane of the surface 200. Further, with the above configuration, a measurement posture becomes a natural and easy posture and thus body movement during the measurement is less frequently generated, so that the blood pressure value can be stably measured.

In the cuff 20 in the present preferred embodiment, the hook 36 and the arm band pull-out port 31d are provided at positions to sandwich the upper arm support surface 31a. Thus, the upper arm 100 can be reliably tightened in the attachment state of the cuff 20 to the upper arm 100. In the cuff 20 in the present preferred embodiment, in a state where the arm band 40 is wound by the winding roller 50 as much as possible, the portion of the arm band 40 close to the other end 41a is pulled out from the arm band pull-out port 31d by a predetermined length, and the pulled portion of the arm band 40 stands up due to the guide portion 34. Thus, a winding task of the arm band 40 can be more easily performed.

In the cuff 20 in the present preferred embodiment, the hole 42 provided in the arm band 40 preferably has a substantially circular shape in a plan view, and the hook 36 provided in the upper arm support stand 30 has a substantially cylindrical shape. Thus, in a state where the arm band 40 is wound around the upper arm 100, the arm band 40 can be slightly rotated taking the hook 36 as a base point. Therefore, even when an outer shape of the upper arm 100 is not a complete cylindrical shape but a truncated conical shape in which a diameter thereof is changed from the center to the edge, the arm band 40 is slightly rotated taking the engaging portion as a base point, so that the arm band is exactly fit for the upper arm 100. Therefore, by adopting the above configuration, the cuff is capable of obtaining a reliable fitting state irrespective of the shape of the upper arm 100.

Further, in the cuff 20 in the present preferred embodiment, the arm band 40 is pulled out from the upper arm support stand 30 by a required length and tightened over the upper arm 100. Thus, the cuff 20 can be attached to the upper arm of anyone who has a larger or smaller outer shape of the upper arm 100 irrespective of size of the diameter of the upper arm 100. That is, the cuff 20 in the present preferred embodiment does not cause the conventional problem in the sphygmomanometer that measurable size of the upper arm is limited by a device configuration and the size of the cuff, and by setting a sufficient length of the arm band 40, the cuff 20 can be reliably attached to the upper arm 100 having a wide difference in size. Therefore, the arm band 40 can be always exactly fit for the upper arm 100 irrespective of a body shape of a user.

As described above, with the sphygmomanometer cuff 20 in the present preferred embodiment, the sphygmomanometer cuff can be easily attached to the upper arm 100 serving as the measurement site having a wide difference in size and reliable winding around the upper arm 100 can be repeated in every measurement. Therefore, with the sphygmomanometer cuff 20 and the sphygmomanometer 1 including the cuff 20, the blood pressure value can be precisely and stably measured, and the measurement can be performed irrespective of the size of the upper arm 100.

It should be noted that the preferred embodiments disclosed herein are illustrative in all aspects and should not be construed as being restrictive. The technical scope of the present invention is defined by the claims, and meanings equivalent to the claims and all modifications within the scope are intended to be encompassed herein.

What is claimed is:

1. A sphygmomanometer cuff attached to an upper arm to be used for measuring a blood pressure value, comprising:
an upper arm support stand arranged to support the upper arm while being mounted on a mount surface; and
a band-shaped arm band arranged to be wound around the upper arm in a state in which the upper arm is supported on the upper arm support stand; wherein
the arm band includes a fluid bag arranged to compress the upper arm while being expanded;
the upper arm support stand includes an upper arm support surface arranged to support the upper arm while the upper arm is placed thereon, a winding mechanism arranged to pull one end in a longitudinal direction of the arm band in a winding direction so as to wind a portion of the arm band close to the one end, and an engaging portion arranged to engage a portion of the arm band close to another end in the longitudinal direction pulled out from the winding mechanism against a pulling force applied by the winding mechanism; and
in a state in which the portion of the arm band close to the another end is engaged with the engaging portion, the upper arm supported on the upper arm support stand is capable of being tightened by the upper arm support surface and the portion of the arm band pulled out from the winding mechanism.

2. The sphygmomanometer cuff according to claim 1, wherein the upper arm support surface is provided in an upper portion of the upper arm support stand while being inclined so that the upper arm support surface is inclined in a state in which the upper arm support stand is mounted on the mount surface.

3. The sphygmomanometer cuff according to claim 1, wherein the upper arm support stand includes an elbow rest on which an elbow is mountable in a state in which the upper arm is placed on the upper arm support surface.

4. The sphygmomanometer cuff according to claim 1, wherein the winding mechanism includes a winding roller to which the one end of the arm band is fixed, and a bias portion arranged to bias the winding roller in the winding direction.

5. The sphygmomanometer cuff according to claim 1, wherein the winding mechanism is arranged inside the upper arm support stand, and the upper arm support stand includes an arm band pull-out port from which the portion of the arm band close to the another end is pulled out.

6. The sphygmomanometer cuff according to claim 5, wherein the engaging portion and the arm band pull-out port are arranged to sandwich the upper arm support surface.

7. The sphygmomanometer cuff according to claim 5, wherein in a state in which the arm band is wound by the winding mechanism as much as possible, the portion of the arm band close to the another end is pulled out from the arm band pull-out port by a predetermined length.

8. The sphygmomanometer cuff according to claim 7, wherein the upper arm support stand includes a guide portion arranged to guide the portion of the arm band close to the another end pulled out from the arm band pull-out port in the state where the arm band is wound by the winding mechanism as much as possible.

9. The sphygmomanometer cuff according to claim 1, wherein the upper arm support stand includes a locking mechanism arranged to prevent pull-out of the arm band from the winding mechanism.

10. The sphygmomanometer cuff according to claim 9, wherein the locking mechanism is actuated in connection with an engagement of the arm band with the engaging portion and is arranged to prevent the pull-out of the arm band from the winding mechanism only in a state in which the arm band is engaged with the engaging portion.

11. The sphygmomanometer cuff according to claim 1, wherein a hole portion is provided in the portion of the arm band close to the another end, and the engaging portion is defined by a hook portion capable of being hung on the hole portion.

12. The sphygmomanometer cuff according to claim 11, wherein the hole portion has a substantially circular shape in a plan view, and the hook portion has a substantially cylindrical shape.

13. A sphygmomanometer, comprising:
the sphygmomanometer cuff according to claim 1;
an expanding/contracting mechanism arranged to expand and contract the fluid bag;
a pressure detector arranged to detect a pressure in the fluid bag; and
a blood pressure value calculating unit arranged to calculate a blood pressure value based on pressure information detected by the pressure detector.
